(19) United States
(12) Patent Application Publication
(10) Pub. No.: US 2019/0167433 A1
(43) Pub. Date: Jun. 6, 2019

(54) ORTHOPEDIC IMPLANT FOR SUSTAINED DRUG RELEASE

(71) Applicant: Duke University, Durham, NC (US)

(72) Inventors: Brian Allen, Durham, NC (US); Kenneth A. Gall, Durham, NC (US); Thorsten M. Seyler, Durham, NC (US); Catherine Oliver, Durham, NC (US)

(21) Appl. No.: 16/196,678

(22) Filed: Nov. 20, 2018

Related U.S. Application Data
(63) Continuation-in-part of application No. 15/973,820, filed on May 8, 2018.
Application No. 15/973,820 is a continuation-in-part of application No. 15/830,561, filed on Dec. 4, 2017, now Pat. No. 10,022,233.

(51) Int. Cl. A61F 2/30; A61F 2/28

(52) U.S. Cl. CPC A61F 2/30721; A61F 2/28; A61F 2002/3068; A61F 2250/0068; A61F 2002/3006; A61F 2002/2835

(57) ABSTRACT

An orthopedic implant device includes an implant body with a reservoir configured store a therapeutic agent. A wall of the implant body has opposite side surfaces, including a side surface facing into the reservoir. Elution channels reach from the reservoir through the body wall. The elution channels include an elongated channel traversing a thickness of the body wall between the opposite side surfaces. The elongated channel may have a length greater than twice the thickness.

### RELATED APPLICATIONS

This application is a continuation-in-part of U.S. patent application Ser. No. 15/973,820, filed May 8, 2018, which is a continuation-in-part of U.S. patent application Ser. No. 15/830,561, filed Dec. 4, 2017, both of which are incorporated by reference.

### TECHNICAL FIELD

This technology relates to an implantable orthopedic device that provides for elution of a therapeutic agent.

### BACKGROUND

An implantable orthopedic device, such as a component of a bone or joint replacement system, may contain an antibiotic or other therapeutic agent for elution from the device while the device is implanted.

### SUMMARY

An orthopedic implant device includes an implant body with a reservoir configured store a therapeutic agent. A wall of the implant body has opposite side surfaces, including a side surface facing into the reservoir. An elution channel reaches from the reservoir through the body wall. The elution channel reaches fully through a thickness of the body wall between the opposite side surfaces, and may have a length that is greater than twice the thickness.

In some examples the elution channel has a length portion reaching within the body wall in a configuration parallel to the opposite side surfaces. Such a length portion may be provided in an arcuate configuration and/or a series of linear sections to define a convoluted elution path through the channel.

The body wall may also have multiple elution channels with a common inlet portion at the side surface facing into the reservoir. The multiple channels may reach from the common inlet portion to different respective outlet portions at the opposite side surface.

In another example, the implant body further has a reinforcement structure, such as a buttress, projecting from the body wall into the reservoir. The channel reaches from the body wall to the reservoir through and within the reinforcement structure.

The reinforcement structure may include as a truss such as, for example, a truss of orthogonal stiffener elements or a diamond cubic truss. Another reinforcement structure may include a minimal surface structure such as a gyroid. The truss or other reinforcement structure may reach across the reservoir fully between opposed portions of the body wall structure that face inward of the reservoir.

In another example, an elution pipe projects from an inner side surface of the body wall into the reservoir. The elution pipe and the body wall together define an elution channel communicating the reservoir with an elution pore in the body wall. The body wall may have a plurality of elution pores, and the elution pipe may be one of a plurality of elution pipes, each of which projects from the inner side surface of the body wall into the reservoir to communicate the reservoir with a respective elution pore.

The implant body wall may further include an adapter for a luer lock fitting to engage a syringe for injecting the therapeutic agent into the reservoir.

### BRIEF DESCRIPTION OF THE DRAWINGS

FIG. 1 is a perspective view of an implantable orthopedic device.

FIG. 2 is a sectional perspective view of a part of the device of FIG. 1.

FIG. 3 is a cross sectional view of the part shown in FIG. 2.

FIG. 4 is a perspective view of another implantable orthopedic device.

FIG. 5 is an opposite perspective view of the device shown in FIG. 4.

FIG. 6 is a perspective view of parts of the device shown in FIGS. 4 and 5.

FIG. 7 is a sectional view of the parts shown in FIG. 6.

FIG. 8 is a partial view of a porous body wall of an implantable orthopedic device.

FIG. 9 is sectional view taken on line 9-9 of FIG. 8.

FIG. 10 is view similar to FIG. 9, shown an alternative porous body wall.

FIG. 11 is a partial view, similar to FIG. 9, of another alternative porous body wall.

FIG. 12 is partial perspective view of an alternative implantable orthopedic device.

FIG. 13 is a side view of the device shown in FIG. 12.

FIGS. 14-16 are partial perspective views of additional alternative devices.

FIG. 17 is a perspective view of part of another alternative device.

FIG. 18 is a sectional view of the part shown in FIG. 17.

FIG. 19 is a view taken on line 19-19 of FIG. 18.

FIG. 20 is a sectional perspective view of another alternative device.

### DETAILED DESCRIPTION

The embodiments illustrated in the drawings have components that are examples of the elements recited in the claims. The illustrated embodiments thus include examples of how a person of ordinary skill in the art can make and use the claimed apparatus. They are described here to meet the enablement and best mode requirements of the patent statute without imposing limitations that are not recited in the claims. One or more elements of one embodiment may be used in combination with, or in substitution for, one or more elements of another embodiment as needed for any particular implementation of the claimed apparatus.

An orthopedic implant device 10 is shown in FIG. 1. This example of an implant device 10 is a tibial component of a total knee replacement system. The device 10 thus includes an implant body 20 including a platform 22 and a stem 24. The platform 22 and the stem 24 are configured to provide elution of a therapeutic agent from within the body 20 over an extended period of time while the device 10 is implanted.

The platform 20 has a peripheral edge surface 30 providing a shape and thickness appropriate for implanting the platform 20 at the proximal end of a tibia. A proximal side surface 32 of the platform 20 serves as a bone-replacement surface, and in this example has a contour configured to replicate a proximal surface contour of a healthy tibial plateau. A distal side surface 34 has a contour configured to mate with the opposed contour of a tibial bone surface that has been surgically prepared to receive the device 10.

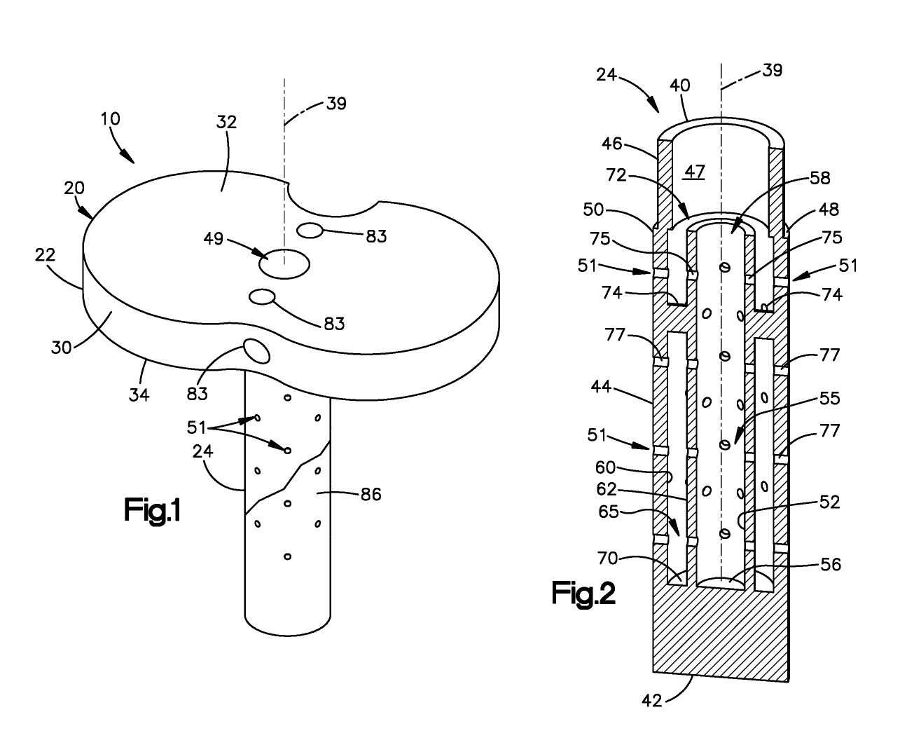

The stem 24 is configured for insertion into the medullary canal of the tibia to anchor the implanted device 10 in place. As best shown in FIG. 2, the stem 24 in the illustrated example has an elongated cylindrical shape with a longitudinal central axis 39, an open proximal end 40, and a closed distal end 42.

A major length section 44 of the stem 24 has a uniform outer diameter. The major length section 44 includes the distal end 42 of the stem 24. A minor length section 46 defines a cylindrical interior space 47, and includes the proximal end 40 of the stem 24. The minor length section 46 also has a reduced outer diameter above a shoulder surface 48. In this manner the minor length section 46 is shaped for fitting into a bore 49 that reaches through the platform 22 to support the stem 24 in the assembled position projecting distally from the platform 22, as shown in FIG. 1.

The major length section 44 of the stem 24 has an exterior surface 50 with pores 51. The major length section 44 further has interior surfaces defining reservoirs and channels in fluid flow communication with the pores 51. These include an innermost cylindrical surface 52 that is centered on the axis 39. The innermost surface 52 defines the length and diameter of a first reservoir 55 having a cylindrical shape reaching along the axis 39 between a closed distal end 56 and an open proximal end 58. A pair of radially opposed cylindrical inner surfaces 60 and 62 also are centered on the axis 39. These inner surfaces 60 and 62 together define the length and width of a second reservoir 65 having an annular shape that is spaced radially outward from, and surrounds, the first reservoir 55. The second reservoir 65 also has a closed distal end 70 and an open proximal end 72. Stiffeners 74 may reach radially across the second reservoir 65 for structural reinforcement.

Additional cylindrical inner surfaces define first and second channels 75 and 77. The first channels 75 reach radially outward from the first reservoir 55 to the second reservoir 65. The second channels 77 reach further outward from the second reservoir 65 to the pores 51. Construction of the reservoirs 55, 65, the channels 75, 77 and the pores 51 is preferably accomplished by an additive manufacturing process that forms the stem 24 as a single unitary body of agglomerated additive manufacturing material.

When the stem 24 is assembled with the platform 22 as shown in FIG. 1, the open proximal ends 58 and 72 of the reservoirs 55, 65 communicate with the bore 49 through the interior space 47 and the open proximal end 40 of the stem 24. Internal channels in the platform 22 may provide fluid flow paths from the bore 49 to additional openings 83.

Before being implanted, the device 10 is charged with a solid therapeutic agent delivery medium. The delivery medium is impregnated with a drug or other therapeutic agent. This can be accomplished by forming a paste-like mixture of the therapeutic agent and a solid binder, and injecting the mixture into the reservoirs 55, 65 through the bore 49 and into the stem 24 through open proximal end 40.

For example, the therapeutic agent may comprise an antibiotic, such as gentamicin, and the solid binder may comprise a powdered material, such as calcium sulfate powder. A paste may be formed by mixing those ingredients with water. As shown partially in FIG. 1, the pores 51 at the exterior surface 50 may be covered with parafilm 86 to contain the injected paste as it solidifies within the reservoirs 55, 65. When the paste has solidified, the parafilm is removed, and the solidified material will then permit gradual elution of the gentamicin outward through the channels 75, 77 from the reservoirs 55, 65, and further outward through the pores 51, as the calcium sulfate delivery medium biodegrades gradually under the influence of the patient's synovial fluid. This sustains the elution over a more extended period of time compared to the more rapid elution of a liquid in the absence of a solid binder.

In addition to the use of a solid binder, the arrangement of reservoirs 55, 65 and channels 75, 77 also contributes to the extended period of time taken for complete elution of the therapeutic agent. Specifically, the channels 75, 77 provide fluid flow communication between the reservoirs 55, 65 in series so that elution from the reservoirs 55, 66 proceeds sequentially rather than simultaneously. Elution is thus sustained as the therapeutic agent in the first reservoir 55 is preserved until the therapeutic agent is depleted or nearly depleted from the second reservoir 65.

Another example of an orthopedic implant device 100 is shown in FIGS. 4 and 5. In this example, the device 100 is a femoral component of a total knee replacement system. Like the device 10 described above, the device 100 is configured to provide elution of a therapeutic agent over an extended period of time.

The device 100 comprises an implant body 110 with medial and lateral legs 112 and 114 that are shaped as medial and lateral condyles. Accordingly, the medial leg 112 has an arcuate shape with a distal end portion 120. The exterior surface 122 at the distal end portion 120 serves as a bone-replacement surface with a contour configured to replicate a healthy medial condyle bone surface contour. The lateral leg 114 similarly has an arcuate shape with a distal end 124 portion at which the exterior surface 126 has a contour replicating a healthy lateral condyle bone surface contour. The distal end portions 120 and 124 are separated across a trochlear gap 125.

An intermediate section 140 of the body 110 reaches across the gap 125 between the medial and lateral legs 112 and 114. The intermediate body section 140 has planar opposite side surfaces 142. Each opposite side surface 142 has an arcuate anterior edge 144 adjoining the adjacent leg 112 or 114. A posterior surface 146 (FIG. 4) has a planar contour reaching across the intermediate body section 140 between the opposite side surfaces 142. An anterior surface 148 (FIG. 5) has an arcuate contour reaching along and across the gap 125 between the legs 112, 114. The posterior and anterior surfaces 146 and 148 each have an array of elution pores 149. In the illustrated example, the all of the elution pores 149 in the body 110 are remote from the bone replacement surface portions 122 and 126.

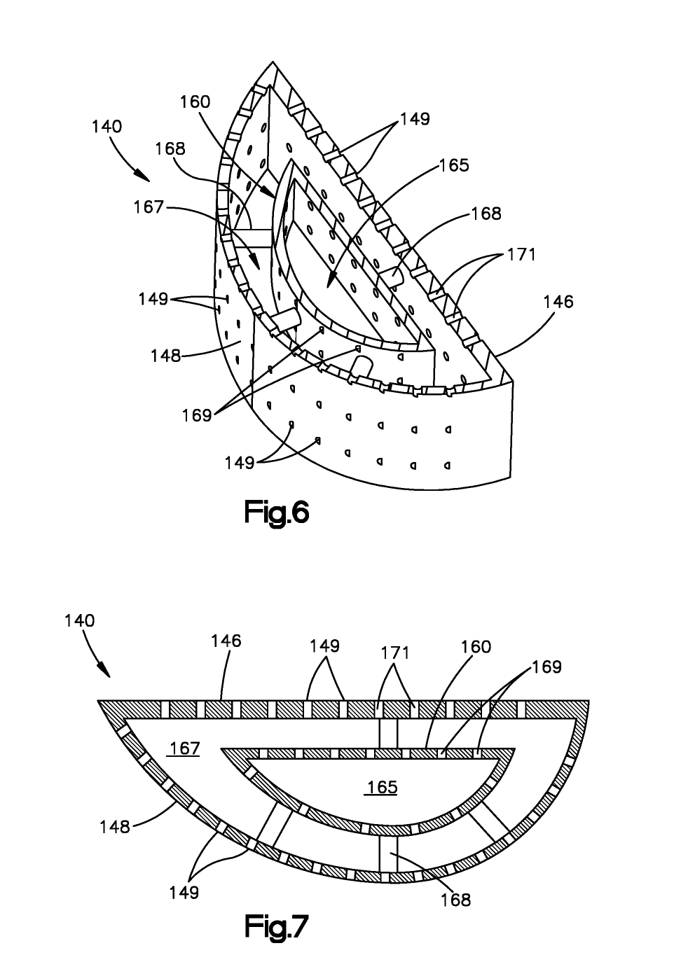

As shown separately in FIGS. 6 and 7, an internal wall structure 160 is located at the interior of the intermediate body portion 140. The internal wall structure 160 divides the interior of into first and second reservoirs 165 and 167. Stiffeners 168 may be provided for structural reinforcement, and the implant body 110 also may be defined by a single unitary body of agglomerated additive manufacturing material.

In use, each reservoir 165 and 167 in the implant body 110 stores a solid therapeutic agent delivery medium impregnated with a therapeutic agent, such as the solidified paste described above. One or more passages for injecting the paste into the reservoirs 165 and 167 can be provided in any suitable manner known in the art of additive manufacturing. Channels 169 reaching through the inner wall structure 160 communicate the first reservoir 165 with the second reservoir 167. Additional channels 171 communicate the second reservoir 167 with the pores 149 at the posterior and anterior external surfaces 146 and 148. The channels 169 and 171 connect the reservoirs 165 and 167 in series so that elution from the reservoirs 165 and 167 to the pores 149 proceeds sequentially rather than simultaneously, whereby elution is sustained as the therapeutic agent in the first reservoir 165 is preserved until the therapeutic agent is depleted or nearly depleted from the second reservoir 167.

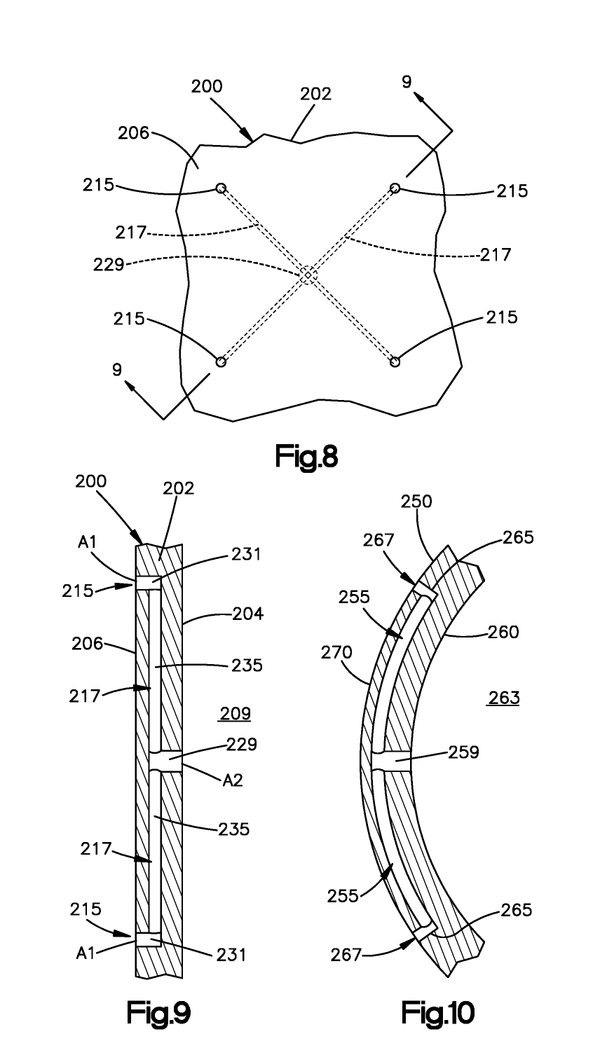

As shown partially in FIGS. 8 and 9, another example of an orthopedic implant device includes an implant body 200 with a body wall 202. The body wall 202 has opposite side surfaces 204 and 206. One side surface 204 faces into a reservoir 209 for storing a solid therapeutic agent delivery medium as described above. The other side surface 206 has elution pores 215 that are spaced apart in an array on that surface 206. Multiple elution channels 217 reach through the body wall 202 to communicate the reservoir 209 with the elution pores 215.

The elution channels 217 in this example have a common inlet portion 229 (FIG. 9) at the reservoir 209. The elution channels 217 also have different respective outlet portions 231 at the elution pores 215. Intermediate portions 235 of the elution channels 217 communicate the inlet portion 229 with the outlet portions 231 in parallel. In this example, the intermediate portions 235 of the elution channels 217 extend fully from the inlet portion 229 to the outlet portions 231 within the thickness of the body wall 202 in linear configurations parallel to the opposite side surfaces 204 and 206.

With the outlet portions 231 of the elution channels 217 spaced apart from the common inlet portion 229, as shown for example in FIGS. 8 and 9, each elution channel 217 has a length that reaches fully through the thickness of the body wall 202 between the opposite side surfaces 204 and 206. Those lengths in the illustrated example are equal, but could alternatively include one or more unequal lengths. The illustrated lengths are also substantially greater than the body wall thickness. Preferably, the length of each elution channel 217 is greater than twice the thickness of the body wall 202, and may be a greater multiple of the thickness, as shown by way of example in FIGS. 8 and 9. This helps to prolong elution from the reservoir 209 to the elution pores 215 for a correspondingly greater period of time.

Additionally, each elution pore 215 in this example has an outlet flow area Al that is substantially less than the common inlet flow area A2. This helps sustain elution by limiting access of the patient's synovial fluid to the solid delivery medium in the reservoir 209. The spaced-apart array of multiple elution pores 215 with a common inlet 229 helps to distribute the therapeutic agent throughout the area of the outer side surface 206, whereas a single outlet would provide a more concentrated delivery of the therapeutic agent.

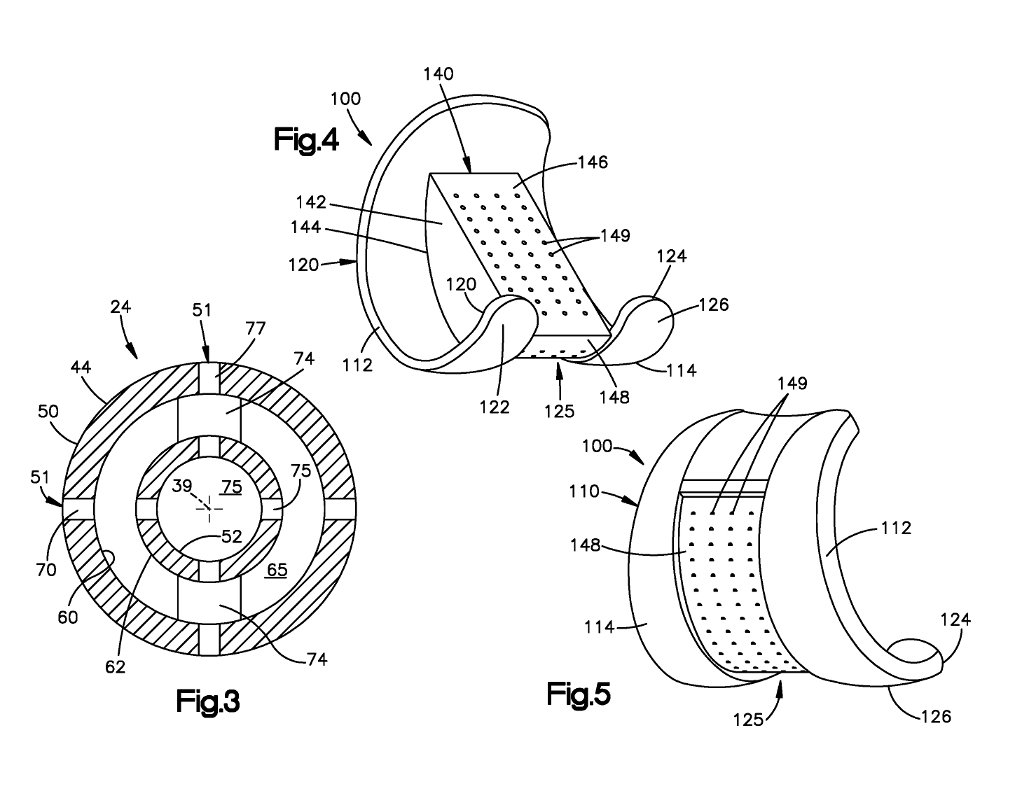

Further regarding the example of FIGS. 8 and 9, the arrangement of elution channels is configured for a flat body wall 200. Such an arrangement could thus be applied to either or both of the flat body walls shown in FIGS. 6 and 7. In another example, a similar arrangement of elution channels on an arcuate body wall 250, as shown in FIG. 10, could be applied to either or both of the arcuate body walls of FIGS. 6 and 7. With a more circular curvature, the arrangement of FIG. 10 could be applied to either or both of the circular body walls shown in FIGS. 2 and 3. In either case, the body wall 250 of FIG. 10 has elution channels 255 with a common inlet portion 259 at a side surface 260 facing into a reservoir 263. The elution channels 225 further have different respective outlet portions 265 that are open at elution pores 267 on an opposite side surface 270. These elution channels 255 extend within the thickness of the body wall 250 fully from the common inlet portion 259 to the outlet portions 255 in arcuate configurations parallel to the opposite side surfaces 260 and 270.

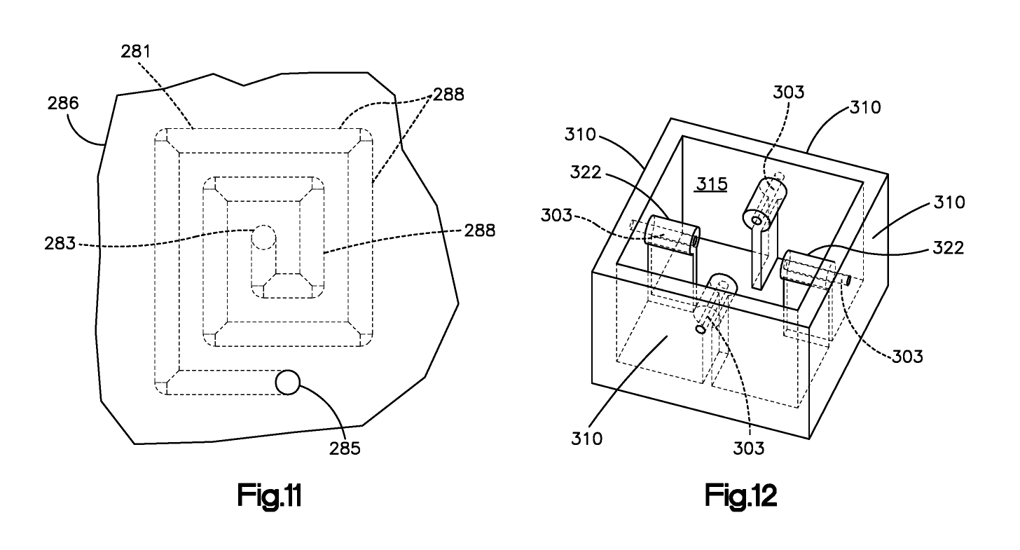

In the example of FIG. 11, an elongated elution channel 281 has an inlet portion 283 and an outlet portion 285. The inlet portion 283 of the channel 281 is located on a porous body wall 286 where an inner side surface of the body wall 286 faces into a reservoir. The outlet portion 285 of the channel 281 is open at an opposite side surface 288 of the body wall 286. As in the examples of FIGS. 8-10, the overall length of the channel 281 in FIG. 11 is greater than twice the thickness of the associated body wall 286 to promote sustained elution. Additionally, the intermediate portion of the channel 281 has a series of linear sections 288 in a non-parallel orientations that provide a convoluted elution flow path between the inlet portion 283 and the outlet portion 285. This further contributes to prolong elution. A similar arrangement can be provided in a spiral or other curvilinear configuration.

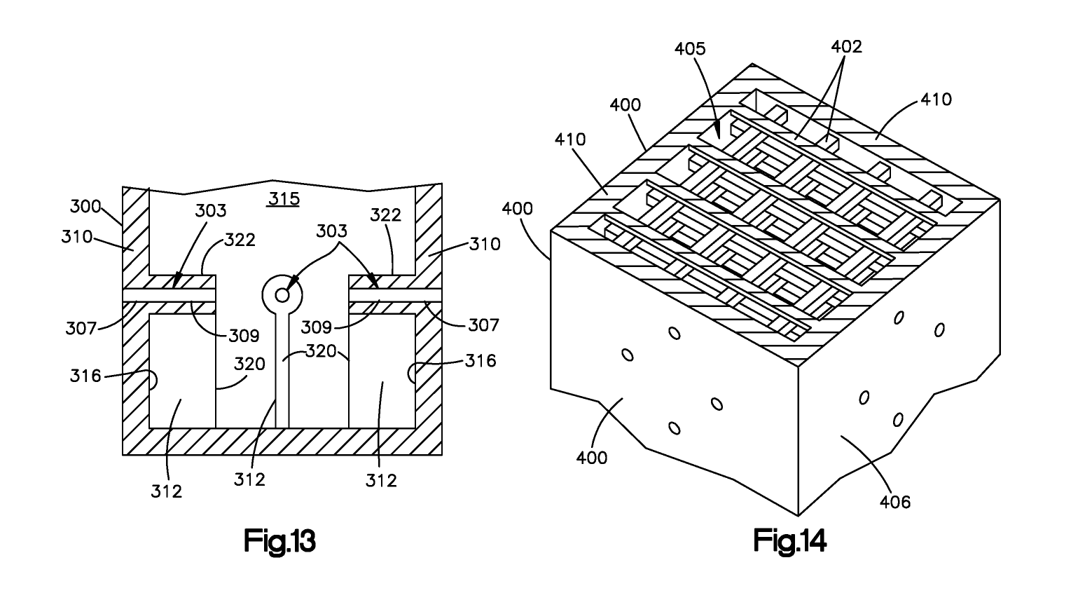

Another example of an implant body 300 with elongated elution channels 303 is shown partially in FIGS. 12 and 13. In this example, each elution channel 303 has a linear configuration with two sections 307 and 309 (FIG. 13). The first section 307 reaches through the associated body wall 310 with a length equal to the surrounding thickness of the body wall 310. The second section 309 provides the channel 303 with a total length 303 that is greater than the body wall thickness by a multiple of two or more. The greater length is provided by configuring the second section 309 of the channel 303 to reach through a buttress 312 that projects from the body wall 310 into the associated reservoir 315. Specifically, the buttress 312 has an edge 316 adjoining the body wall 310, and reaches from the adjoined edge 316 to a free edge 320 within the reservoir 315. The second section 308 of the channel 303 reaches along and through an enlarged-width portion 322 of the buttress 312. The enlarged width portion 322 in the illustrated example is configured as a pipe.

In the example of FIGS. 12 and 13, the buttresses 312 provide structural reinforcement to the body wall portions that are rendered porous by the elution channels 303. This can enable the body walls 310 to have decreased wall thickness and/or increased porosity.

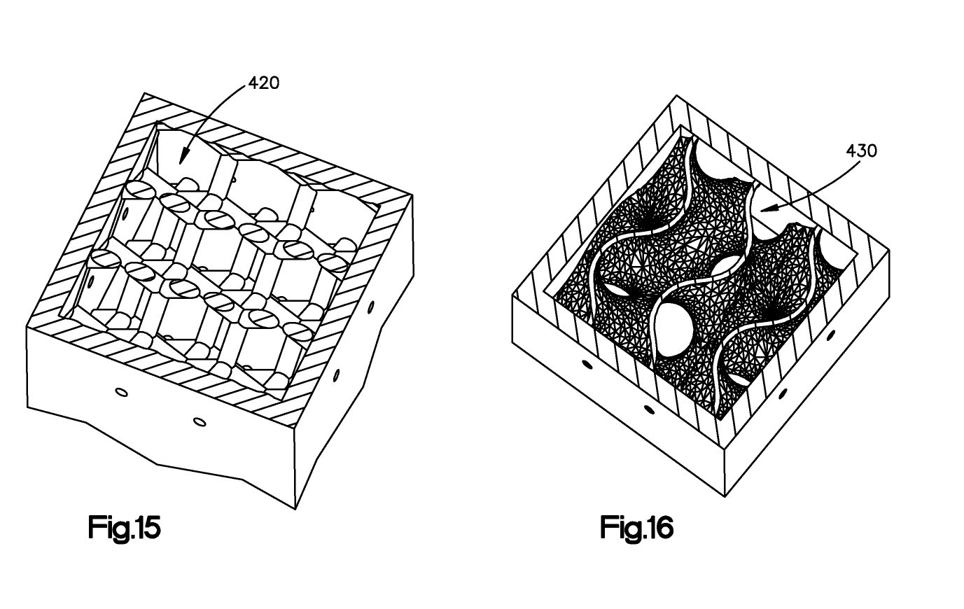

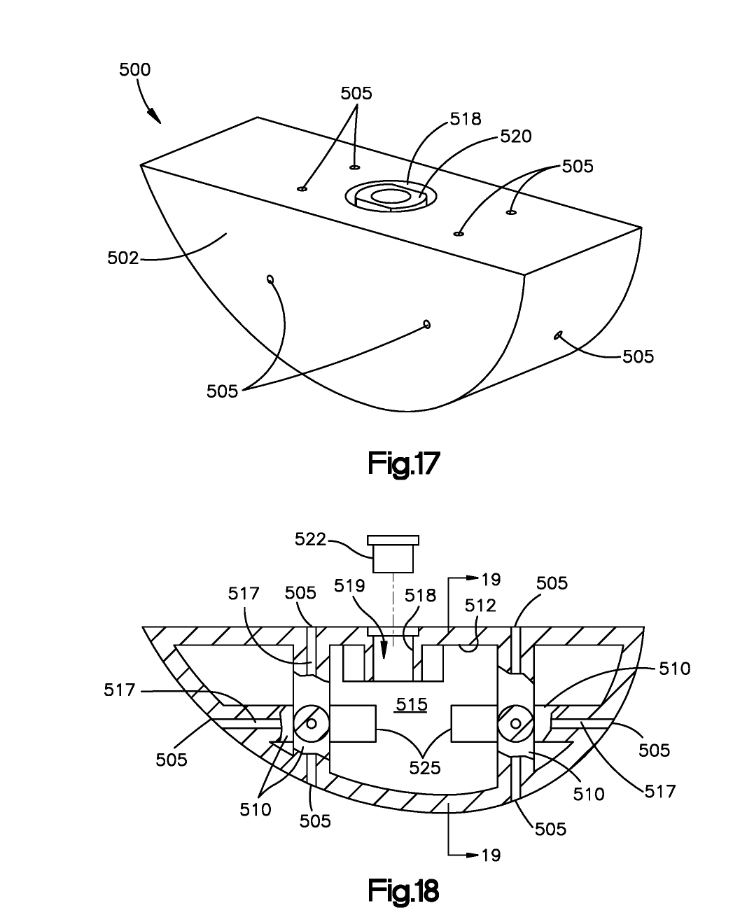

Structural reinforcement can also be provided in other configurations, as shown for example in FIGS. 14, 15 and 16. In the example of an implant body 400 as shown FIG. 14, structural reinforcement is provided by a truss of stiffener elements 402. The stiffener elements 402 in this example are configured as beams in an orthogonal array reaching across the reservoir 405 fully from porous body wall portions 406 to opposed body wall portions 410. In the example of FIG. 15, a truss 420 is provided in the configuration of a diamond cubic truss. In the example of FIG. 16, structural reinforcement is provided by a minimal surface structure in the configuration of a gyroid. Each of these examples of a reinforcement structure 402, 420 and 430 also projects across the respective reservoir fully from a porous body wall portion to an opposed body wall portion that faces inward of the reservoir. Such structures can be formed within the surrounding body wall structure by use of know additive manufacturing techniques.

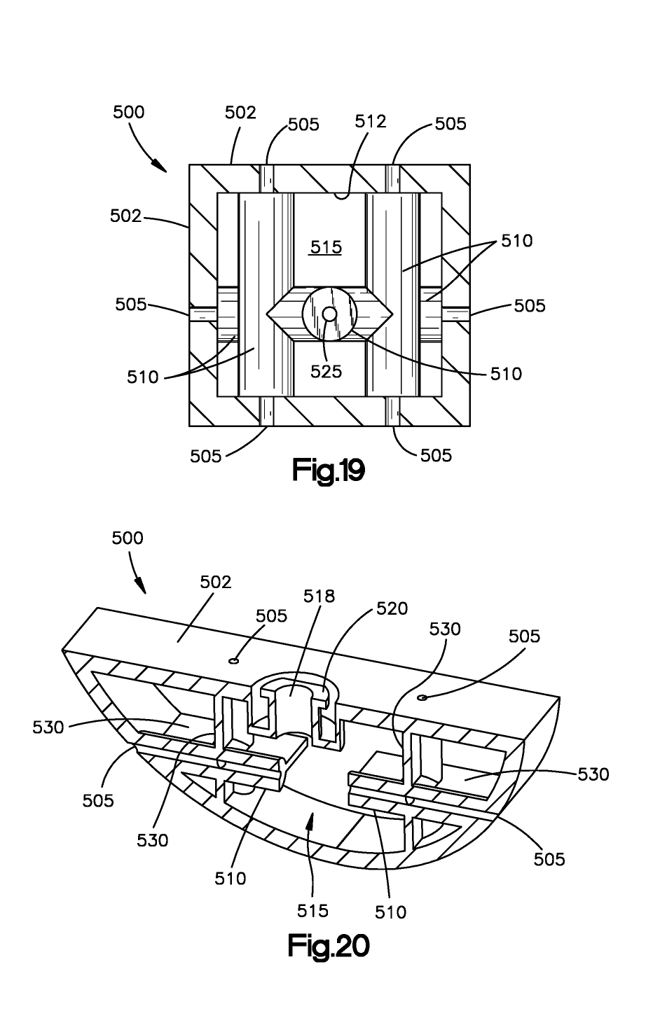

FIG. 17 is a partial view of an implant body wall 500 similar to that shown in FIG. 6. The body wall 500 of FIG. 17 also has a surface 502 with elution pores 505. As further shown in FIGS. 18 and 19, elution pipes 510 project from an opposite side surface 512 of the body wall 500 into a reservoir 515 for containing a therapeutic delivery agent. The elution pipes 510 and the body wall 502 together define elution channels 517 that provide fluid communication between the reservoir 515 and the elution pores 505.

The body wall 500 further includes an adapter 518 for a luer lock fitting to secure a syringe for injecting the therapeutic agent delivery medium agent into the reservoir 515 as described above. The adapter 518 defines a passage 519 into the reservoir 515 and, in the given example, has a male flange 520 for receiving and guiding an internal screw thread on a female luer fitting. A closure device 522 in the form of a plug or cap 522 is provided for closing and sealing the passage 519.

The elution pipe 510 in the example of FIGS. 17-19 are arranged in two separate sets. Each set includes a single inlet pipe 510 with an inlet 525 inside the reservoir 515. Each set further includes multiple branch pipes 510 that reach from the inlet pipe 510 to respective elution pores 505. The pipes 510 in each set thus share a common inlet 525 within the reservoir 515.

FIG. 20 shows a variation of the example shown in FIGS. 17-19. As shown partially in FIG. 20, reinforcement structures in the form of buttresses 530 are provided to reach from the pipes 510 to the body wall 500. These buttresses 530 are formed as inner walls with planar opposite sides, and reach lengthwise along the pipes 510 as shown.

This written description sets for the best mode of carrying out the invention, and describes the invention so as to enable a person of ordinary skill in the art to make and use the invention, by presenting examples of the elements recited in the claims. The detailed descriptions of those elements do not impose limitations that are not recited in the claims, either literally or under the doctrine of equivalents.

What is claimed is:

1. An orthopedic implant device comprising:
an implant body including:
a reservoir configured to store a therapeutic agent; and
a body wall having opposite side surfaces including a side surface facing into the reservoir, and further having an elution channel reaching from the reservoir through the body wall;
wherein the elution channel reaches fully through a thickness of the body wall between the opposite side surfaces, and the elution channel has a length that is greater than twice the thickness.

2. An orthopedic implant device as defined in claim 1, wherein the channel has a length portion reaching within the porous body wall in a configuration parallel to the opposite side surfaces.

3. An orthopedic implant device as defined in claim 2, wherein the configuration is linear.

4. An orthopedic implant device as defined in claim 2, wherein the configuration is arcuate.

5. An orthopedic implant as defined in claim 1, wherein the implant body further has a reinforcement structure projecting from the body wall into the reservoir, and the channel reaches from the reservoir to the body wall through and within the reinforcement structure.

6. An orthopedic implant as defined in claim 5, wherein the reinforcement structure comprises a buttress projecting from the body wall into the reservoir.

7. An orthopedic implant device as defined in claim 1, wherein the channel is one of multiple channels, each of which traverses a respective thickness of the body between the opposite side surfaces with a length greater than twice the respective thickness.

8. An orthopedic implant device as defined in claim 7, wherein the multiple channels are of equal length.

9. An orthopedic implant device as defined in claim 7, wherein the multiple channels include channels of unequal length.

10. An orthopedic implant device as defined in claim 7, wherein the multiple channels have a common inlet portion at the reservoir, and reach from the common inlet portion to different respective outlet portions.

11. An orthopedic implant device as defined in claim 1 further comprising a solid therapeutic agent delivery medium that is stored in the reservoir and is biodegradable under the influence of synovial fluid, and a therapeutic agent infused within the solid therapeutic agent delivery medium.

12. An orthopedic implant device as defined in claim 1, wherein the implant body further includes a bone-replacement surface with a contour configured to replicate a healthy bone surface contour.

13. An orthopedic implant device as defined in claim 12, wherein the contour of the bone-replacement surface is configured to replicate a healthy tibial bone surface contour.

14. An orthopedic implant device as defined in claim 12, wherein the contour of the bone-replacement surface is configured to replicate a healthy femoral bone surface contour.

15. An orthopedic implant device comprising:
an implant body including:
a reservoir configured to store a therapeutic agent; and
a body wall having opposite side surfaces including a side surface facing into the reservoir, and further having elution channels reaching through the body wall between the opposite side surfaces;
wherein the elution channels include multiple channels having a common inlet portion at the side surface facing into the reservoir, and the multiple channels reach from the common inlet portion to different respective outlet portions at an opposite side surface.

16. An orthopedic implant device as defined in claim 15, wherein the multiple channels include a channel having a linear configuration between the inlet portion and the respective outlet portion.

17. An orthopedic implant device as defined in claim 15, wherein the multiple channels include a channel having an arcuate configuration between the inlet portion and the respective outlet portion.

18. An orthopedic implant device as defined in claim 15, wherein the multiple channels are of equal length.

19. An orthopedic implant device as defined in claim 15, wherein the multiple channels include channels of unequal length.

20. An orthopedic implant device as defined in claim 15, wherein the common inlet portion of the multiple channels has a first fluid flow area, and the outlet portions include an outlet portion having a fluid flow area less that the fluid flow area of the common inlet portion.

21. An orthopedic implant device as defined in claim 15, wherein the outlet portions include multiple outlet portions having fluid flow areas less that the fluid flow area of the common inlet portion.

22. An orthopedic implant device as defined in claim 17, further comprising a solid therapeutic agent delivery medium that is stored in the reservoir and is biodegradable under the influence of synovial fluid, and a therapeutic agent infused within the solid therapeutic agent delivery medium.

23. An orthopedic implant device as defined in claim 17, wherein the implant body further includes a bone-replacement surface with a contour configured to replicate a healthy bone surface contour.

24. An orthopedic implant device as defined in claim 23, wherein the contour of the bone-replacement surface is configured to replicate a healthy tibial bone surface contour.

25. An orthopedic implant device as defined in claim 23, wherein the contour of the bone-replacement surface is configured to replicate a healthy femoral bone surface contour.

26. An orthopedic implant device comprising:
an implant body including:
a reservoir configured to store a therapeutic agent;
a body wall facing into the reservoir and having an elution channel reaching from the reservoir through the body wall; and
a reinforcement structure projecting from the body wall into the reservoir.

27. An orthopedic implant device as defined in claim 26, wherein the reinforcement structure comprises a buttress projecting from the body wall into the reservoir.

28. An orthopedic implant device as defined in claim 27, wherein the buttress has an edge adjoining the body wall, and has a free edge within the reservoir, and the elution channel reaches through and within the buttress from the body wall to the free edge within the reservoir.

29. An orthopedic implant device as defined in claim 26, wherein the body wall is part of an inwardly facing body wall structure having portions opposed across the reservoir, and the reinforcement structure reaches across the reservoir fully between opposed portions of the inwardly facing body wall structure.

30. An orthopedic implant device as defined in claim 29, wherein the reinforcement structure is configured as a gyroid.

31. An orthopedic implant device as defined in claim 29, wherein the reinforcement structure is a truss.

32. An orthopedic device as defined in claim 31, wherein the truss includes orthogonal stiffener elements.

33. An orthopedic device as defined in claim 31, wherein the truss is a diamond cubic truss.

34. An orthopedic implant device as defined in claim 26, further comprising a solid therapeutic agent delivery medium that is stored in the reservoir and is biodegradable under the influence of synovial fluid, and a therapeutic agent infused within the solid therapeutic agent delivery medium.

35. An orthopedic implant device as defined in claim 26, wherein the implant body further includes a bone-replacement surface with a contour configured to replicate a healthy bone surface contour.

36. An orthopedic implant device as defined in claim 35, wherein the contour of the bone-replacement surface is configured to replicate a healthy tibial bone surface contour.

37. An orthopedic implant device as defined in claim 35, wherein the contour of the bone-replacement surface is configured to replicate a healthy femoral bone surface contour.

38. An orthopedic implant device comprising:
an implant body including:
a reservoir configured to store a therapeutic agent;
a body wall having an inner side surface facing into the reservoir and an opposite side surface with an elution pore; and
an elution pipe that projects from the inner side surface of the body wall into the reservoir, wherein the elution pipe and the body wall together define an elution channel communicating the reservoir with the elution pore.

39. An orthopedic implant device as defined in claim 38, wherein the body wall has a plurality of elution pores, and the elution pipe is one of a plurality of elution pipes, each of which projects from the inner side surface of the body wall into the reservoir and communicates the reservoir with a respective elution pore.

40. An orthopedic implant device as defined in claim 39, wherein the elution pipes include elution pipes having a common inlet within the reservoir.

41. An orthopedic implant device as defined in claim 38, further comprising a reinforcement structure including buttresses interconnecting the body wall with the elution pipes.

42. An orthopedic implant device as defined in claim 38, wherein the implant body further includes a bone-replacement surface with a contour configured to replicate a healthy bone surface contour.

43. An orthopedic implant device as defined in claim 42, wherein the contour of the bone-replacement surface is configured to replicate a healthy tibial bone surface contour.

44. An orthopedic implant device as defined in claim 42, wherein the contour of the bone-replacement surface is configured to replicate a healthy femoral bone surface contour.

45. An orthopedic implant device comprising:
an implant body including:
a reservoir configured to store a therapeutic agent; and
a body wall facing into the reservoir and having an elution channel reaching from the reservoir through the body wall;
wherein the body wall includes an adapter defining a passage through the body wall for injection of a therapeutic agent into the reservoir, and the adapter is configured for engagement with a luer lock fitting.

46. An orthopedic implant device as defined in claim 45, wherein the adapter includes a male flange configured to receive and guide an internal screw thread on a female luer lock fitting.

47. An orthopedic device as defined in claim 45, further comprising a closure device configured to close the passage through the adapter.

48. An orthopedic implant device as defined in claim 45, further comprising a solid therapeutic agent delivery medium that is stored in the reservoir and is biodegradable under the influence of synovial fluid, and a therapeutic agent infused within the solid therapeutic agent delivery medium.

49. An orthopedic implant device as defined in claim 45, wherein the implant body further includes a bone-replacement surface with a contour configured to replicate a healthy bone surface contour.

50. An orthopedic implant device as defined in claim 49, wherein the contour of the bone-replacement surface is configured to replicate a healthy tibial bone surface contour.

51. An orthopedic implant device as defined in claim 49, wherein the contour of the bone-replacement surface is configured to replicate a healthy femoral bone surface contour.
